(19) United States
(12) Patent Application Publication
(10) Pub. No.: US 2025/0325459 A1
(43) Pub. Date: Oct. 23, 2025

(54) COSMETIC COMPOSITION COMPRISING AMINO ACIDS, HYDROXYLATED (POLY)CARBOXYLIC ACIDS AND POLYSACCHARIDES, PROCESSES AND USE

(71) Applicant: L'OREAL, Paris (FR)

(72) Inventors: Manon CHAUMONTET, SAINT-OUEN (FR); Patrick MINOU, SAINT-OUEN (FR); Laura FENELON, SAINT-OUEN (FR)

(21) Appl. No.: 18/870,013

(22) PCT Filed: May 31, 2023
(86) PCT No.: PCT/EP2023/064584

§ 371 Date: Nov. 27, 2024

(30) Foreign Application Priority Data
Jun. 1, 2022 (FR) FR2205255

(51) Int. Cl. A61K 8/44; A61K 8/365; A61K 8/73; A61Q 5/00

(52) U.S. Cl. CPC A61K 8/44; A61K 8/365; A61K 8/737; A61Q 5/002

(57) ABSTRACT

The present invention relates to a cosmetic composition, preferably a hair composition, comprising at least 0.8% by weight of compounds of amino acid type, one or more hydroxylated polycarboxylic acids, comprising 2 to 8 carbon atoms and at least 3 COOH groups, and/or salts thereof, and one or more galactomannans.

The invention also relates to a cosmetic treatment process, in particular for washing and/or conditioning the hair, using said composition.

The present invention relates to a cosmetic composition, notably a hair composition, comprising one or more compounds of amino acid type, one or more hydroxycarboxylic acids and one or more polysaccharides. The invention also relates to a cosmetic treatment process using said composition.

Consumers worldwide are generally in contact with highly varied sources of water which are not without impact on the hair, notably with regard to their cosmetic properties, and/or the performance qualities of hair products.

“Mineral” waters contain, for example, variable amounts of minerals present in the form of dissolved ions, such as calcite (present in the form of calcium), dolomite (present in the form of calcium and magnesium), magnetite (present in the form of iron) and chalcanthite (present in the form of copper). “Hard” waters are also concentrated in minerals, such as calcium and magnesium, and swimming pool waters are for their part concentrated in copper salts originating from algicides used in the treatment of swimming pools.

Hair has a strong tendency to absorb these minerals and/or their metal salts because of the presence, at the surface thereof, of anionic functional groups which correspond in particular to the sulfonic or carboxylic functional groups of keratin. Furthermore, the isoelectric point of the individual hair is generally described between 3.2 and 4. The result of this is that, in everyday life, the pH of the water applied to the hair is greater than such values, which results in a negatively charged fibre.

Minerals, very often polyvalent cations, will thus be attracted and captured by this negatively charged fibre with the formation of chemical bonds, which prevent them from being released by conventional hair treatment processes. This results in a possible accumulation of minerals on the hair with the passage of time. Such an attaching depends not only on the hardness of the water, on the frequency and/or duration of exposure of the hair to the water in question but also on the nature and the length of the hair (in particular porosity and charge) and also on its state of damage.

The accumulation of these minerals and/or of their metal salts can result in modifications to the hair fibre and in particular in a more or less marked modification of the cosmetic properties of the individual hair. Thus, an accumulation of calcium and of magnesium can result in dry hair lacking in gloss, whereas an accumulation of copper can result in the hair turning green.

In addition, the accumulation of the metal (iron, copper, for example) salts can accelerate the damage caused to the hair because they catalyse oxidation/reduction reactions and generate hydroxyl radicals HO° which can be harmful to the keratin fibre, including at low contents.

This can result in a photodegradation of the fibre, in a lightening of the fibre, and also in a detrimental change in the properties of the hair, which can result in premature breakage of the individual hair; these phenomena are very particularly observed during the subsequent use of lightening products or dyeing products.

In other words, the hair can become less resistant, more weakened, indeed even break more easily, or also lose its sheen, due to the accumulation of minerals and/or their metal salts.

There thus exists a real need to have available compositions which make it possible to combat the accumulation of the metal ions, resulting from minerals and metal salts dissolved in water, indeed even to make it possible to extract them from keratins fibres, in order to limit their negative impacts and to overcome all of the abovementioned disadvantages.

The composition which is the subject of the present invention, and the use thereof, make it possible to achieve this aim.

One subject of the present invention is thus a cosmetic composition, preferably a hair composition, comprising:
one or more compounds of amino acid type chosen from the compounds corresponding to formula (I) below and/or salts thereof, present in a total content of at least 0.8% by weight, relative to the total weight of the composition,
one or more hydroxylated polycarboxylic acids, comprising from 2 to 8 carbon atoms, and at least 3 COOH groups, and/or salts thereof, preferably present in a total content of at least 0.5% by weight, relative to the total weight of the composition, and
one or more polysaccharides chosen from galactomannans including their derivatives.

It has been found that the composition according to the invention makes it possible to improve the resistance to breakage of the hair, to strengthen the hair and also to significantly limit its reduction or loss of sheen, undesirable effects liable to be caused by the presence of metal ions, notably copper or calcium ions, within said fibres.

After application of the composition, the fibres appear to be strengthened, said strengthening being improved with successive applications of the composition.

It has also been found that the composition according to the invention also makes it possible to contribute conditioning properties to the hair, notably a smooth feel, softness, sheen and facilitated disentangling, and to do so while contributing strength, body and a bulk effect to the head of hair.

It finds a very particular application in the cosmetic treatment, notably the washing and/or conditioning, of keratin fibres that have been sensitized, weakened and/or damaged, notably as a result of physical (repeated brushing) and/or chemical treatments, for example dyeing, bleaching, perming and/or straightening.

It is particularly suitable for the cosmetic treatment, notably the washing and/or conditioning, of keratin fibres laden with metals, notably with calcium and/or copper in contents of at least 100 ppm, better still in contents of at least 200 ppm; notably laden with copper, notably in contents of at least 100 ppm, better still in contents of at least 200 ppm and/or laden with calcium notably in contents of at least 4000 ppm, better still in contents of at least 10 000 ppm.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”, and may be replaced therewith.

### Compounds of Amino Acid Type

The composition according to the present invention comprises one or more compounds of amino acid type, chosen from the compounds corresponding to formula (I) below and/or salts thereof.

The compounds of amino acid type may thus correspond to formula (I):

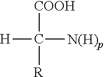

in which p is an integer equal to 1 or 2, it being understood that:
when p=1, R forms, with the nitrogen atom, a saturated heterocycle comprising from 5 to 8 ring members, preferably 5 ring members, it being possible for this ring to be substituted with one or more groups chosen from hydroxyl or (C₁-C₄)alkyl;
when p=2, R represents a hydrogen atom or a saturated, linear or branched, (C₁-C₁₂)alkyl, preferably (C₁-C₄)alkyl, group, optionally interrupted with one or more heteroatoms or groups chosen from —S—, —NH— or —C(NH)— and/or optionally substituted with one or more groups chosen from hydroxyl (—OH), amino (—NH₂), —SH, —COOH, —CONH₂ or —NH—C(NH)—NH₂.

Preferably, when p=1, R forms, with the nitrogen atom, a saturated heterocycle comprising 5 ring members, this ring not being substituted.

Preferably, p=2.

Preferably, when p=2, R represents a hydrogen atom or a saturated, linear or branched, (C₁-C₄)alkyl group, optionally interrupted with a —S— heteroatom and/or optionally substituted with one or two groups chosen from hydroxyl, amino or —NH—C(NH)—NH₂.

Preferentially, p=2 and R represents a hydrogen atom.

The compounds of amino acid type may also be a salt of a compound of formula (I). These salts comprise the salts with organic or mineral bases, for example the salts of alkali metals, for instance the lithium, sodium or potassium salts; the salts of alkaline-earth metals, for instance the magnesium or calcium salts, and the zinc salts.

The compounds of amino acid type may be in the form of an optical isomer of L, D or DL configuration, preferably of L configuration.

As examples according to the present invention of compounds in the form of an optical isomer of L configuration, mention may be made of L-proline, L-methionine, L-serine, L-arginine and L-lysine.

Preferably, the compound(s) of amino acid type according to the invention are chosen from glycine, proline, methionine, serine, arginine, lysine, their salts (notably alkali metal, alkaline-earth metal or zinc salts) and mixtures thereof.

Preferably, the compound(s) of amino acid type according to the invention are chosen from glycine, proline, methionine, serine, arginine, salts thereof and mixtures thereof.

Better still, the compound of amino acid type is chosen from glycine, salts thereof (notably alkali metal, alkaline-earth metal or zinc salts) and mixtures thereof.

As glycine salts according to the present invention, mention may be made of sodium glycinate, zinc glycinate, calcium glycinate, magnesium glycinate, manganese glycinate and potassium glycinate, preferably sodium glycinate and potassium glycinate.

Preferably, the compound of amino acid type is glycine.

The total content of compound(s) of amino acid type present in the composition according to the invention is at least 0.8% by weight, relative to the total weight of the composition. This content may range from 0.8% to 10% by weight, notably from 0.9% to 8% by weight, better still from 0.95% to 7% by weight, or even from 1% to 6% by weight, relative to the total weight of the composition.

In particular, the total content of compound(s) of aminocarboxylic acid type in the composition according to the invention may range from 0.8% to 10% by weight, notably from 0.9% to 8% by weight, better still from 0.95% to 7% by weight, or even from 1% to 6% by weight, relative to the total weight of the composition.

Better still, the total content of compound(s) of amino acid type chosen from glycine, proline, methionine, serine, arginine, lysine, salts thereof and mixtures thereof in the composition according to the invention may range from 0.8% to 10% by weight, notably from 0.9% to 8% by weight, better still from 0.95% to 7% by weight, or even from 1% to 6% by weight, relative to the total weight of the composition.

Most particularly, the total content of compound(s) of amino acid type chosen from glycine, salts thereof and mixtures thereof in the composition according to the invention may range from 0.8% to 10% by weight, notably from 0.9% to 8% by weight, better still from 0.95% to 7% by weight, or even from 1% to 6% by weight, relative to the total weight of the composition.

Better still, the glycine content in the composition according to the invention may range from 0.8% to 10% by weight, notably from 0.9% to 8% by weight, better still from 0.95% to 7% by weight, or even from 1% to 6% by weight, relative to the total weight of the composition.

### Hydroxypolycarboxylic Acids

The composition according to the invention also comprises one or more hydroxylated polycarboxylic acids comprising from 2 to 8 carbon atoms and at least 3 COOH groups, and/or salts thereof.

These polycarboxylic acids are different from the compounds of amino acid type described above.

Said polyacids comprise at least 3 COOH groups (in acid or salified form), better 3 COOH groups (in acid or salified form).

They also comprise at least one OH group but may comprise several thereof, notably from two to three OH groups.

Preferably, they comprise in total from four to six carbon atoms and their hydrocarbon-based chain is saturated and linear.

Advantageously, the hydroxylated polycarboxylic acids and/or salts thereof comprise in total from four to six carbon atoms, from one to three OH groups and three COOH groups (in acid or salified form).

The salts of these polyacids comprise the salts with organic or mineral bases, for example the salts of alkali metals, such as the lithium, sodium or potassium salts; the salts of alkaline-earth metals, such as the magnesium or calcium salts, and the zinc salts. The alkali metal or alkaline-earth metal salts are preferred and in particular the sodium salts.

Preferably, the hydroxylated polycarboxylic acids or salts thereof are chosen from α-hydroxy acids and salts thereof, and notably from citric acid and salts thereof, notably alkali metal or alkaline-earth metal salts such as sodium citrate.

Preferably, the total content of hydroxylated polycarboxylic acids comprising in total from two to eight carbon atoms and at least 3 COOH groups, and/or salts thereof, present in the composition according to the invention is at least 0.5% by weight, relative to the total weight of the composition. This content may range from 0.5% to 10% by weight, notably from 1% to 8% by weight, better still from 1.2% to 6% by weight, even better still from 1.5% to 5% by weight, relative to the total weight of the composition.

In particular, the total content of hydroxylated polycarboxylic acids comprising in total from four to six carbon atoms, from one to three OH groups and three COOH groups, or salts thereof, present in the composition according to the invention may range from 0.5% to 10% by weight, notably from 1% to 8% by weight, better still from 1.2% to 6% by weight, even better still from 1.5% to 5% by weight, relative to the total weight of the composition.

Most particularly, the total content of hydroxylated polycarboxylic acids chosen from citric acid and salts thereof, notably alkali metal or alkaline-earth metal salts, in the composition according to the invention may range from 0.5% to 10% by weight, notably from 1% to 8% by weight, better still from 1.2% to 6% by weight, even better still from 1.5% to 5% by weight, relative to the total weight of the composition.

### Polysaccharides

The composition according to the invention also comprises one or more polysaccharides chosen from galactomannans including their derivatives; they may advantageously be chosen from nonionic galactomannans, cationic galactomannans, their derivatives, notably their ethers or esters, and mixtures thereof.

The composition could additionally comprise one or more polysaccharides different from said galactomannans, for example chosen from celluloses, starches and their derivatives, notably their ethers or esters, and mixture thereof. They may advantageously be chosen from nonionic celluloses, cationic celluloses, nonionic starches, and mixtures thereof.

These polymers may be physically or chemically modified. Mention may be made, as physical treatment, of the temperature and mention may be made, as chemical treatment, of esterification, etherification, amidation and oxidation reactions, in so far as these treatments make it possible to give polymers that are nonionic.

As galactomannans that may be used, mention may be made of nonionic guar gums which can be modified with (poly)hydroxy(C₁-C₆)alkyl groups, notably hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups. The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are, for example, sold under the trade names Jaguar HP8, Jaguar HP60, Jaguar HP120, Jaguar HP105 SGI and Jaguar HP8 SGI by the company Rhodia Chimie.

The cationic galactomannans (or cationic galactomannan gums) are notably described in U.S. Pat. Nos. 3,589,578 and 4,031,307; mention may be made of cationic guar gums, notably those comprising cationic trialkylammonium groups, notably trimethylammonium. Mention may thus be made of guar gums modified with a 2,3-epoxypropyltrimethylammonium salt (for example a chloride).

Preferably, 2% to 30% by number of the hydroxyl functions of the guar gums bear cationic trialkylammonium groups. Even more preferentially, 5% to 20% by number of the hydroxyl functions of these guar gums are branched with cationic trialkylammonium groups. Among these trialkylammonium groups, mention may most particularly be made of the trimethylammonium and triethylammonium groups. Even more preferentially, these groups represent from 5% to 20% by weight relative to the total weight of the modified guar gum. According to the invention, guar gums modified with 2,3-epoxypropyltrimethylammonium chloride may be used.

Mention may be made of the products having the INCI names Hydroxypropyl guar hydroxypropyltrimonium chloride and Guar hydroxypropyltrimonium chloride. Such products are notably sold under the names Jaguar C13S, Jaguar C15, Jaguar C17 and Jaguar C162 by the company Solvay.

The botanical origin of the starch molecules that may optionally be used may be cereals or tubers. Thus, the starches are chosen, for example, from corn starch, rice starch, cassava starch, barley starch, potato starch, wheat starch, sorghum starch and pea starch. The starches may be chemically or physically modified, notably by one or more of the following reactions: pregelatinization, oxidation, crosslinking, esterification, etherification, amidation, heat treatments.

The starch molecules may be derived from any plant source of starch, in particular such as corn, potato, oat, rice, tapioca, sorghum, barley or wheat. It is also possible to use hydrolysates of the starches mentioned above. The starch is preferably derived from potato.

As nonionic cellulose that may optionally be used, mention may be made of cellulose-based polymers not including a C₁₀-C₃₀ fatty chain in their structure.

The term “cellulose-based” refers to any polysaccharide compound bearing in its structure sequences of glucose residues linked together by β-1,4 bonds; the cellulose-based polymers may be unsubstituted celluloses, and/or derivatives of nonionic celluloses.

Thus, the cellulose-based polymers that may be used may be chosen from unsubstituted celluloses, including those in a microcrystalline form, and cellulose ethers. Among these cellulose-based polymers, cellulose ethers, cellulose esters and cellulose ether/esters are distinguished.

Among the nonionic cellulose ethers that may be mentioned are (C₁-C₄)alkylcelluloses, such as methylcelluloses and ethylcelluloses (for example Ethocel Standard 100 Premium from Dow Chemical); (poly)hydroxy(C₁-C₄)alkylcelluloses, such as hydroxymethylcelluloses, hydroxyethylcelluloses (for example Natrosol 250 HHR sold by Aqualon) and hydroxypropylcelluloses (for example Klucel EF from Aqualon); mixed (poly)hydroxy(C₁-C₄)alkyl(C₁-C₄)alkylcelluloses, such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the optional cationic celluloses, mention may also be made of cellulose ether derivatives including quaternary ammonium groups, cationic cellulose copolymers or cellulose derivatives grafted with a water-soluble quaternary ammonium monomer.

The cellulose ether derivatives including quaternary ammonium groups are notably described in FR 1 492 597; they are also defined in the CTFA dictionary as quaternary ammoniums of hydroxyethylcellulose that has reacted with an epoxide substituted with a trimethylammonium group.

Mention may notably be made of the polymers sold under the name Ucare Polymer JR (JR 400 LT, JR 125 and JR 30M) or LR (LR 400 and LR 30M) by the company Amerchol.

Cationic cellulose copolymers and cellulose derivatives grafted with a water-soluble quaternary ammonium monomer are described notably in patent U.S. Pat. No. 4,131,576; mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. Mention may be made most particularly of crosslinked or non-crosslinked quaternized hydroxyethylcelluloses, the quaternizing agent notably possibly being diallyldimethylammonium chloride; and most particularly hydroxypropyltrimethylammonium hydroxyethylcellulose.

Among the commercial products corresponding to this definition, mention may be made of the products sold under the names Celquat L 200 and Celquat H 100 by the company National Starch.

A particularly preferred cationic cellulose that may notably be mentioned is the polymer having the INCI name Polyquaternium-10.

Among the optional cationic polysaccharides that may be used, mention may also be made of cationic derivatives of cassia gum, notably those including quaternary ammonium groups; in particular, mention may be made of the product having the INCI name Cassia hydroxypropyltrimonium chloride.

Preferably, the galactomannans which may be used in the context of the invention are chosen, alone or as a mixture, from cationic galactomannan gums, notably cationic guar gums; nonionic guar gums optionally modified with (poly)hydroxy(C₁-C₆)alkyl groups, notably hydroxypropyl; and mixtures thereof; better from nonionic guar gums optionally modified with (poly)hydroxy(C₁-C₆)alkyl, notably hydroxypropyl (INCI name: Hydroxypropyl Guar), groups.

Preferably, the composition additionally comprises one or more polysaccharides chosen from cationic celluloses such as Polyquaternium-10; nonionic celluloses, which may or may not be substituted, such as nonionic cellulose ethers such as (C1-C4)alkylcelluloses and (poly)hydroxy(C1-C4)alkylcelluloses; and mixture thereof.

In a preferred embodiment, the composition according to the invention comprises:
one or more nonionic guar gums optionally modified with C₁-C₆ (poly)hydroxylalkyl groups, notably hydroxypropyl, and
one or more cationic polysaccharides notably chosen from cationic celluloses such as Polyquaternium-10 and/or cationic galactomannan gums, notably cationic guar gums.

The composition according to the invention may comprise the polysaccharide(s) (galactomannan(s) and optional polysaccharide(s) if any) in a total amount ranging from 0.01 to 10% by weight, better still from 0.05 to 5% by weight, still better still from 0.1 to 2% by weight, relative to the total weight of the composition.

The composition according to the invention may comprise galactomannan(s) in a total amount ranging from 0.01 to 10% by weight, better still from 0.05 to 5% by weight, still better still from 0.1 to 2% by weight, relative to the total weight of the composition.

The composition according to the invention may comprise polysaccharide(s) chosen, alone or as a mixture, from cationic celluloses; cationic galactomannan gums, notably cationic guar gums; nonionic guar gums optionally modified with C1-C6 (poly)hydroxylalkyl groups, notably hydroxypropyl; and/or nonionic celluloses, which may or may not be substituted, such as nonionic cellulose ethers such as (C1-C4)alkylcelluloses and (poly)hydroxy(C1-C4)alkylcelluloses; in a total amount ranging from 0.01 to 10% by weight, better still from 0.05 to 5% by weight, still better still from 0.1 to 2% by weight, relative to the total weight of the composition.

Notably, the composition according to the invention may comprise the cationic polysaccharide(s) in a total amount ranging from 0.01% to 10% by weight, better still from 0.05% to 5% by weight, even better still from 0.1% to 2% by weight, relative to the total weight of the composition.

Notably, the composition according to the invention may comprise the nonionic polysaccharide(s) in a total amount ranging from 0.01% to 10% by weight, better still from 0.05% to 5% by weight, even better still from 0.1% to 2% by weight, relative to the total weight of the composition.

Notably, the composition according to the invention may comprise the cationic galactomannan(s) in a total amount ranging from 0.01% to 10% by weight, better still from 0.05% to 5% by weight, even better still from 0.1% to 2% by weight, relative to the total weight of the composition.

Notably, the composition according to the invention may comprise the nonionic galactomannan(s) in a total amount ranging from 0.01% to 10% by weight, better still from 0.05% to 5% by weight, even better still from 0.1% to 2% by weight, relative to the total weight of the composition.

### Nonionic Surfactants

The composition according to the invention may optionally comprise one or more nonionic surfactants.

Examples of nonionic surfactants that may be mentioned include the following compounds, alone or as a mixture:
oxyalkylenated (C₈-C₂₄)alkylphenols;
saturated or unsaturated, linear or branched, oxyalkylenated or glycerolated C₈-C₄₀ alcohols, preferably including one or two fatty chains;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
preferably oxyethylenated esters of saturated or unsaturated, linear or branched, C₈ to C₃₀ acids and of sorbitol;
fatty acid esters of sucrose;
optionally oxyalkylenated alkyl (poly)glycosides (0 to 10 oxyalkylene units) which may comprise from 1 to 15 glucose units;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
amine oxides.

The oxyalkylene units are more particularly oxyethylene or oxypropylene units, or a combination thereof, preferably oxyethylene units.

The number of moles of ethylene oxide and/or propylene oxide preferably ranges from 1 to 250, more particularly from 2 to 100 and better still from 2 to 50; the number of moles of glycerol ranges notably from 1 to 50 and better still from 1 to 10. Advantageously, the nonionic surfactants according to the invention do not comprise any oxypropylene units.

Preferably, they comprise a number of moles of ethylene oxide ranging from 1 to 250, notably from 2 to 100 and better still from 2 to 50.

As examples of glycerolated nonionic surfactants, use is preferably made of monoglycerolated or polyglycerolated C₈ to C₄₀ alcohols, comprising from 1 to 50 mol of glycerol and preferably from 1 to 10 mol of glycerol.

Mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleyl/cetyl alcohol containing 6 mol of glycerol, and octadecanol containing 6 mol of glycerol.

Among the glycerolated alcohols, it is more particularly preferred to use the C₈ to C₁₀ alcohol containing 1 mol of glycerol, the C₁₀ to C₁₂ alcohol containing 1 mol of glycerol and the C₁₂ alcohol containing 1.5 mol of glycerol.

Nonionic surfactants of alkyl (poly)glycoside type may notably be represented by the following general formula: R1O—(R2O)t-(G)v in which:
R1 represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms,
R2 represents an alkylene radical including 2 to 4 carbon atoms;
G represents a sugar unit including 5 to 6 carbon atoms;
t denotes a value ranging from 0 to 10 and preferably from 0 to 4;
v denotes a value ranging from 1 to 15 and preferably from 1 to 4.

Preferably, the alkyl(poly)glycoside surfactants are compounds of the formula described above in which:
R1 denotes a linear or branched, saturated or unsaturated alkyl radical including from 8 to 18 carbon atoms,
R2 represents an alkylene radical including 2 to 4 carbon atoms;
t denotes a value ranging from 0 to 3 and preferably equal to 0,
G denotes glucose, fructose or galactose, preferably glucose,
it being possible for the degree of polymerization, i.e. the value of v, to range from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C8/C16 alkyl (poly)glucosides of 1-4 type, and notably decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of C8/C16 alkyl (poly)glycosides of 1-4 type, notably as an aqueous 53% solution, such as those sold by Cognis under the reference Plantacare® 818 UP.

The nonionic surfactant(s) used in the composition according to the invention are preferentially chosen, alone or as a mixture, from:
saturated or unsaturated, linear or branched, oxyethylenated C₈ to C₄₀ alcohols comprising from 1 to 100 mol of ethylene oxide, preferably from 2 to 50 and more particularly from 2 to 40 mol of ethylene oxide and preferably including one or two fatty chains;
saturated or unsaturated oxyethylenated plant oils comprising from 1 to 100 and preferably from 2 to 50 mol of ethylene oxide;
(C₈-C₃₀)alkyl (poly)glycosides which are optionally oxyalkylenated, preferably with from 0 to 10 mol of ethylene oxide, and which comprise from 1 to 15 glucose units;
monoglycerolated or polyglycerolated C₈ to C₄₀ alcohols, comprising from 1 to 50 mol of glycerol and preferably from 1 to 10 mol of glycerol;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
preferably oxyethylenated esters of saturated or unsaturated, linear or branched, C₈ to C₃₀ acids and of sorbitol;

More preferentially, the nonionic surfactant(s) used in the composition according to the invention are chosen, alone or as a mixture, from:
saturated or unsaturated, linear or branched, oxyethylenated C₈ to C₄₀ alcohols comprising from 1 to 100 mol of ethylene oxide, preferably from 2 to 50 and more particularly from 2 to 40 mol of ethylene oxide and including one or two fatty chains, notably at least one C₈-C₂₀, notably C₁₀-C₁₈, alkyl chain,
preferably oxyethylenated esters of saturated or unsaturated, linear or branched, C₈ to C₃₀ acids and of sorbitol, and
(C₈-C₃₀)alkyl(poly)glucosides, which are optionally oxyalkylenated, preferably comprising from 0 to 10 mol of ethylene oxide and comprising 1 to 15 glucose units.

Preferably, the composition according to the invention comprises the nonionic surfactant(s) in a total content ranging from 0.05% to 10% by weight, preferably from 0.1% to 5% by weight, preferentially from 0.2% to 3% by weight, relative to the total weight of the composition according to the invention.

### Cationic Surfactants

The composition according to the invention may optionally comprise one or more cationic surfactants.

They are preferably chosen from primary, secondary or tertiary fatty amines, which are optionally polyoxyalkylenated, or salts thereof, and quaternary ammonium salts, and mixtures thereof.

The composition can comprise one or more cationic surfactants chosen, alone or as a mixture, from the following compounds, which are quaternary ammonium salts:
the compounds corresponding to the general formula (II) below:

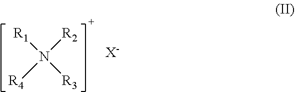

in which:
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates, the groups R₁ to R₄, which may be identical or different, represent a linear or branched aliphatic group including from 1 to 30 carbon atoms, or an aromatic group such as aryl or alkylaryl, at least one of the groups R₁ to R₄ denoting a linear or branched aliphatic group including from 8 to 30 carbon atoms, preferably from 12 to 24 carbon atoms.

The aliphatic groups may include heteroatoms notably such as oxygen, nitrogen, sulfur and halogens. The aliphatic groups are chosen, for example, from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Among the quaternary ammonium salts of formula (II), the ones that are preferred are tetraalkylammonium salts, for instance dialkyldimethylammonium or alkyltrimethylammonium salts in which the alkyl group includes from about 12 to 22 carbon atoms, in particular behenyltrimethylammonium, distearyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts, or stearamidopropyldimethyl(myristyl acetate)ammonium salts such as those sold under the name Ceraphyl® 70 by the company Van Dyk.

It is preferred in particular to use the chloride, bromide or methyl sulfate salts of these compounds.
the quaternary ammonium salts of imidazoline, such as those of formula (III):

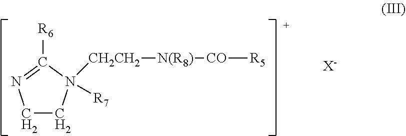

in which R5 represents an alkenyl or alkyl group including from 8 to 30 carbon atoms, derived for example from tallow fatty acids, R₆ represents a hydrogen atom, a C₁-C₄ alkyl group or an alkyl or alkenyl group including from 8 to 30 carbon atoms, R7 represents a C₁-C₄ alkyl group, R8 represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms.

Preferably, R5 and R6 denote a mixture of alkenyl or alkyl groups including from 12 to 21 carbon atoms, for example derived from tallow fatty acids, R7 denotes a methyl group and R8 denotes a hydrogen atom.

Such a product is sold, for example, under the name Rewoquat® W 75 by the company Rewo,
quaternary diammonium or triammonium salts, in particular of formula (IV):

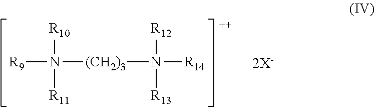

in which:
R₉ denotes an alkyl radical including from about 16 to 30 carbon atoms which is optionally hydroxylated and/or optionally interrupted with one or more oxygen atoms,
R₁₀ is chosen from hydrogen or an alkyl radical including from 1 to 4 carbon atoms or a group (R₉ₐ)(R₁₀ₐ)(R₁₁ₐ)N—(CH₂)₃, with R₉ₐ, R₁₀ₐ, R₁₁ₐ, R₁₁, R₁₂, R₁₃ and R₁₄, which may be identical or different, chosen from hydrogen or an alkyl radical including from 1 to 4 carbon atoms, and
X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, sold by Finetex (Quaternium 89), and Finquat CT, sold by Finetex (Quaternium 75);
quaternary ammonium salts containing at least one ester function, such as those of formula (V) below:

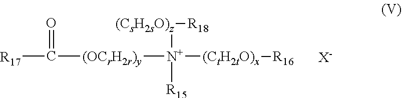

in which:
R₁₅ is chosen from C₁-C₆ alkyl groups and C₁-C₆ hydroxyalkyl or dihydroxyalkyl groups;
R₁₆ is chosen from the group R19-C(O)—; groups R₂₀ which are linear or branched, saturated or unsaturated C₁-C₂₂ hydrocarbon-based groups; a hydrogen atom;
R₁₈ is chosen from the group R21-C(O)—; groups R₂₂ which are linear or branched, saturated or unsaturated C₁-C₆ hydrocarbon-based groups; a hydrogen atom;
R₁₇, R₁₉ and R₂₁, which may be identical or different, are chosen from linear or branched, saturated or unsaturated C₇-C₂₁ hydrocarbon-based groups;
r, s and t, which may be identical or different, are integers having values from 2 to 6;
y is an integer ranging from 1 to 10;
x and z, which may be identical or different, are integers having a value from 0 to 10;
X⁻ is a simple or complex, organic or mineral anion;
with the proviso that the sum x+y+z is from 1 to 15, that when x is 0 then R₁₆ denotes R₂₀, and that when z is 0 then R₁₈ denotes R₂₂.

The alkyl groups R₁₅ may be linear or branched, and more particularly linear. Preferably, R₁₅ denotes a methyl, ethyl, hydroxyethyl or dihydroxypropyl group, and more particularly a methyl or ethyl group.

Advantageously, the sum x+y+z is from 1 to 10.

When R₁₆ is a hydrocarbon-based group R₂₀, it may be long and contain from 12 to 22 carbon atoms, or may be short and contain from 1 to 3 carbon atoms.

When R₁₈ is a hydrocarbon-based group R₂₂, it preferably contains 1 to 3 carbon atoms.

Advantageously, R₁₇, R₁₉ and R₂₁, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function. The anion X⁻ is even more particularly chloride or methyl sulfate.

In the composition according to the invention, use may be made more particularly of the ammonium salts of formula (V) in which R₁₅ denotes a methyl or ethyl group, x and y are equal to 1; z is equal to 0 or 1; r, s and t are equal to 2;
R₁₆ is chosen from the group R19-C(═O)—, methyl, ethyl or C₁₄-C₂₂ hydrocarbon-based groups, and a hydrogen atom;
R₁₈ is chosen from the group R21-C(═O)— and a hydrogen atom;
R₁₇, R₁₉ and R₂₁, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Mention may be made, for example, of the compounds of formula (V) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (notably chloride or methyl sulfate), and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in U.S. Pat. Nos. 4,874,554 and 4,137,180.

Use may be made of behenoylhydroxypropyltrimethylammonium chloride sold by KAO under the name Quatarmin BTC 131.

Preferably, the ammonium salts containing at least one ester function contain two ester functions.

Among the quaternary ammonium salts containing at least one ester function that may be used, it is preferred to use dipalmitoylethylhydroxyethylmethylammonium salts.

The term “fatty amine” means a compound comprising at least one optionally (poly)oxyalkylenated primary, secondary or tertiary amine function, or salts thereof and comprising at least one C₆-C₃₀ and preferably C₈-C₃₀ hydrocarbon-based chain.

Preferably, the fatty amines of use according to the invention are not (poly)oxyalkylenated.

Fatty amines that may be mentioned include amidoamines. The amidoamines according to the invention may be chosen from fatty amidoamines, it being possible for the fatty chain to be borne by the amine group or by the amido group.

The term “amidoamine” means a compound comprising at least one amide function and at least one primary, secondary or tertiary amine function.

The term “fatty amidoamine” means an amidoamine comprising, in general, at least one C₆-C₃₀ hydrocarbon-based chain. Preferably, the fatty amidoamines of use according to the invention are not quaternized.

Preferably, the fatty amidoamines of use according to the invention are not (poly)oxyalkylenated.

Mention may be made, among the fatty amidoamines of use according to the invention, of the amidoamines of following formula (VI):

RCONHR″N(R′)₂ (VI)

in which:
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C₅-C₂₉ and preferably C₇-C₂₃ alkyl radical, or a linear or branched C₅-C₂₉ and preferably C₇-C₂₃ alkenyl radical;
R″ represents a divalent hydrocarbon-based radical containing less than 6 carbon atoms, preferably from 2 to 4 carbon atoms and better still 3 carbon atoms; and
R′, which may be identical or different, represent a substituted or unsubstituted, saturated or unsaturated, linear or branched, monovalent hydrocarbon-based radical containing less than 6 carbon atoms, preferably from 1 to 4 carbon atoms, preferably a methyl radical.

The fatty amidoamines of formula (VI) are chosen, for example, from oleamidopropyldimethylamine, stearamidopropyldimethylamine, notably the product sold by the company Inolex Chemical Company under the name Lexamine S13, isostearamidopropyldimethylamine, stearamidoethyldimethylamine, lauramidopropyldimethylamine, myristamidopropyldimethylamine, behenamidopropyldimethylamine, dilinoleamidopropyldimethylamine, palmitamidopropyldimethylamine, ricinoleamindopropyldimethylamine, soyamidopropyldimethylamine, avocadoamidopropyldimethylamine, cocamidopropyldimethylamine, minkamidopropyldimethylamine, oatamidopropyldimethylamine, sesamidopropyldimethylamine, tallamidopropyldimethylamine, olivamidopropyldimethylamine, palm itamidopropyldimethylamine, stearamidoethyldiethylamine, brassicamidopropyldimethylamine and mixtures thereof.

Preferably, the fatty amidoamines are chosen from oleamidopropyldimethylamine, stearamidopropyldimethylamine, brassicamidopropyldimethylamine and mixtures thereof.

The cationic surfactant(s) are preferably chosen from those of formula (II), those of formula (V), those of formula (VI), and mixtures thereof; better still from those of formula (II) and/or of formula (VI); even better still from those of formula (II). Preferentially, the cationic surfactant(s) may be chosen from salts such as chlorides, bromides or methosulfates, of tetraalkylammonium, for instance dialkyldimethylammonium or alkyltrimethylammonium salts in which the alkyl group includes from about 12 to 22 carbon atoms, in particular behenyltrimethylammonium, distearyldimethylammonium, cetyltrimethylammonium or benzyldimethylstearylammonium salts; dipalmitoylethylhydroxyethylmethylammonium salts such as dipalmitoylethylhydroxyethylmethylammonium methosulfate; and mixtures thereof.

Even more preferentially, they are chosen from cetyltrimethylammonium chloride, behenyltrimethylammonium chloride, dipalmitoylethylhydroxyethylmethylammonium methosulfate, and mixtures thereof.

Preferably, the composition according to the invention may comprise the cationic surfactant(s) in a total amount ranging from 0.1% to 15% by weight, better still from 0.2% to 10% by weight, preferentially from 0.5% to 8% by weight, even better still from 1% to 6% by weight, relative to the total weight of the composition.

### Fatty Substance

The composition according to the invention may optionally comprise one or more non-silicone fatty substances, which may be chosen from solid fatty substances, liquid fatty substances and mixtures thereof.

The term “non-silicone fatty substance” means a fatty substance not containing any Si—O bonds.

The term “solid fatty substance” means a fatty substance having a melting point of greater than 25° C., preferably greater than or equal to 28° C., preferentially greater than or equal to 30° C., at atmospheric pressure (1.013×10⁵ Pa).

Advantageously, the solid fatty substances that may be used in the present invention are neither (poly)oxyalkylenated nor (poly)glycerolated.

The solid fatty substances may be chosen from solid fatty acids, solid fatty alcohols, solid esters of fatty acids and/or of fatty alcohols, waxes and ceramides, and mixtures thereof.

The term “fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated. The solid fatty acids that may be used in the present invention are notably chosen from myristic acid, cetylic acid, stearylic acid, palmitic acid, stearic acid, lauric acid, behenic acid, and mixtures thereof. Said fatty acids are different from the (poly)hydroxylated carboxylic acids comprising from 2 to 8 carbon atoms described previously.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated. The solid fatty alcohols may be saturated or unsaturated, and linear or branched, and include from 8 to 40 carbon atoms, preferably from 10 to 30 carbon atoms, better still from 12 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 12 to 30, or even from 12 to 24 atoms and even better still from 14 to 22 carbon atoms. The solid fatty alcohols that may be used are preferably chosen from saturated, and linear or branched, preferably linear and saturated, (mono)alcohols including from 8 to 40 carbon atoms, better still from 10 to 30, or even from 12 to 24 atoms and better still from 14 to 22 carbon atoms.

The solid fatty alcohols that may be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol or their mixtures, such as cetylstearyl alcohol; better still, the solid fatty alcohol is cetylstearyl alcohol.

The solid esters of a fatty acid and/or of a fatty alcohol that may be used are preferably chosen from the esters resulting from C₉-C₂₆ fatty carboxylic acid and/or from C₉-C₂₆ fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C₄-C₂₂ dicarboxylic or tricarboxylic acids and of C₁-C₂₂ alcohols and esters of monocarboxylic, dicarboxylic or tricarboxylic acids and of C₂-C₂₆ dihydroxy, trihydroxy, tetrahydroxy or pentahydroxy alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl, cetyl or stearyl palmitate; C₉-C₂₆ alkyl myristates, such as cetyl myristate, stearyl myristate and myristyl myristate; and C₉-C₂₆ alkyl stearates, notably myristyl, cetyl and stearyl stearate; and mixtures thereof. Particularly preferably, the solid esters of a fatty acid and/or of a fatty alcohol are chosen from myristyl stearate, myristyl palmitate and mixtures thereof.

For the purposes of the present invention, a wax is a lipophilic compound, which is solid at 25° C. and atmospheric pressure, with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may notably be made of hydrocarbon-based waxes, for instance beeswax or modified beeswaxes (cera bellina), lanolin wax and lanolin derivatives, spermaceti; cork fiber or sugarcane waxes, olive tree wax, rice bran wax, carnauba wax, candelilla wax, ouricury wax, esparto grass wax, berry wax, shellac wax, Japan wax and sumac wax, absolute waxes of flowers; montan wax, orange wax, lemon wax, microcrystalline waxes, paraffins, petroleum jelly, lignite and ozokerite; polyethylene waxes, the waxes obtained by Fischer-Tropsch synthesis and waxy copolymers, and also esters thereof.

Mention may also be made of C₂₀ to C₆₀ microcrystalline waxes, such as Microwax HW.

Mention may also be made of the MW 500 polyethylene wax sold under the reference Permalen 50-L Polyethylene.

Mention may also be made of the waxes obtained by catalytic hydrogenation of animal or plant oils containing linear or branched C₈-C₃₂ fatty chains. Among these waxes, mention may notably be made of isomerized jojoba oil such as trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, notably the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may also be used is a C20 to C40 alkyl (hydroxystearyloxy)stearate (the alkyl group comprising from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may notably be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter, shea butter or cork fibre or sugar cane waxes, olive tree wax, rice bran wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

Ceramides, or ceramide analogues, such as glycoceramides, that may be used in the compositions according to the invention, are known; mention may in particular be made of ceramides of classes I, II, III and V according to the Dawning classification.

The ceramides or analogues thereof that may be used preferably correspond to the following formula:

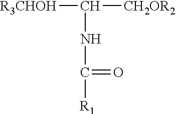

in which:
R1 denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R2 denotes a hydrogen atom or a (glycosyl)n group, a (galactosyl)m group or a sulfogalactosyl group, in which n is an integer ranging from 1 to 4 and m is an integer ranging from 1 to 8;
R3 denotes a C₁₅-C₂₆ hydrocarbon-based group which is saturated or unsaturated in the alpha position, it being possible for this group to be substituted with one or more C₁-C₁₄ alkyl groups;
it being understood that, in the case of natural ceramides or glycoceramides, R3 can also denote a C15-C26 α-hydroxyalkyl group, the hydroxyl group being optionally esterified with a C16-C30 α-hydroxy acid.

Preferentially, ceramides are used for which R1 denotes a saturated or unsaturated alkyl group derived from C14-C30 fatty acids; R2 denotes a galactosyl or sulfogalactosyl group; and R3 denotes a —CH═CH—(CH2)12-CH3 group.

The ceramides that are more particularly preferred are the compounds for which R1 denotes a saturated or unsaturated alkyl derived from C16-C22 fatty acids; R2 denotes a hydrogen atom; and R3 denotes a saturated or unsaturated linear C15 group.

Use may also be made of the compounds for which R₁ denotes a saturated or unsaturated alkyl radical derived from C₁₂-C₂₂ fatty acids; R2 denotes a galactosyl or sulfogalactosyl radical and R3 denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol: 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

As liquid fatty substances that may be used, mention may be made of liquid hydrocarbons, liquid fatty alcohols, liquid esters of fatty acids and/or fatty alcohols other than the triglycerides, oils of triglyceride type of plant or synthetic origin, mineral oils and mixtures thereof.

The liquid fatty substances have a melting point of less than or equal to 25° C., preferably of less than or equal to 20° C., at atmospheric pressure (1.013×10⁵ Pa).

Advantageously, the liquid fatty substances are not (poly)oxyalkylenated.

It is recalled that the fatty alcohols, esters and acids more particularly contain at least one saturated or unsaturated, linear or branched hydrocarbon-based group comprising from 6 to 40 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

The liquid hydrocarbons may be C₆ to C₁₈ liquid hydrocarbons and be linear, branched or optionally cyclic; they are preferably chosen from C₈-C₁₆, notably C₁₀-C₁₄, alkanes. Examples that may be mentioned include hexane, cyclohexane, undecane, dodecane, isododecane, tridecane or isoparaffins, such as isohexadecane or isodecane, and mixtures thereof.

The liquid hydrocarbons may also be chosen from those comprising more than 16 carbon atoms, which may be linear or branched, of mineral or synthetic origin; mention may be made of liquid paraffins or liquid petroleum jelly, polydecenes, hydrogenated polyisobutene, such as Parleam®, and mixtures thereof.

The triglyceride oils of plant or synthetic origin may be chosen from liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearinerie Dubois or those sold under the names Miglyol® 810, 812 and 818 by the company Dynamit Nobel, jojoba oil and shea butter oil, and mixtures thereof.

The liquid fatty alcohols may be chosen from linear or branched, saturated or unsaturated alcohols, preferably unsaturated or branched alcohols, including from 6 to 40 carbon atoms and preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or fatty alcohols other than the triglycerides mentioned above, mention may notably be made of esters of saturated or unsaturated, linear C₁ to C₂₆ or branched C₃ to C₂₆, aliphatic mono- or polyacids and of saturated or unsaturated, linear C₁ to C₂₆ or branched C₃ to C₂₆, aliphatic mono- or polyalcohols, the total carbon number of the esters being greater than or equal to 6, more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, 2-ethylhexyl palmitate, 2-octyldecyl palmitate; alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate or isopropyl palmitate, alkyl myristates, such as isopropyl or ethyl myristate, isocetyl stearate, 2-ethylhexyl isononanoate, isodecyl neopentanoate, isostearyl neopentanoate and mixtures thereof.

Still within the context of this variant, esters of C₄-C₂₂ dicarboxylic or tricarboxylic acids and of C₁-C₂₂ alcohols and esters of monocarboxylic, dicarboxylic or tricarboxylic acids and of C₂-C₂₆ dihydroxy, trihydroxy, tetrahydroxy or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C6 to C30 and preferably C12 to C22 fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described above and of linear or branched, saturated or unsaturated C6 to C30 and preferably C12 to C22 fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters.

More particularly, use is made of monoesters and diesters and notably sucrose, glucose or methylglucose mono- or di-oleates, -stearates, -behen-ates, -oleopalmitates, -linoleates, -linolenates and -oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

Preferably, the fatty substances are chosen from triglyceride oils of plant or synthetic origin, liquid esters of a fatty acid and/or a fatty alcohol other than triglycerides, liquid C₆-C₁₈ hydrocarbons, solid fatty alcohols, solid esters of fatty acids and/or of fatty alcohols, and mixtures thereof.

Preferably, the composition according to the invention may comprise the fatty substance(s) in a total amount ranging from 0.1% to 20% by weight, better still from 1% to 18% by weight, preferentially from 2% to 15% by weight, even better still from 5% to 12% by weight, relative to the total weight of the composition.

### Additional Compounds

The composition according to the invention advantageously comprises water, notably in a concentration preferably ranging from 50% to 95% by weight, for example from 55% to 90% by weight, notably from 60% to 85% by weight, better still from 65% to 85% by weight, relative to the total weight of the composition.

The pH of the composition may be between 2.5 and 8, preferentially between 3 and 7, or even between 4 and 6.

The composition according to the invention may optionally comprise one or more preferably hydrophilic (water-soluble or water-miscible) organic solvents which are liquid at 25° C., 1 atm, which may be chosen from C₁-C₆ aliphatic or aromatic monoalcohols, C₂-C₈ polyols and C₃-C₇ polyol ethers. Advantageously, the organic solvent is chosen from C₂-C₄ mono-, di- or tri-diols. It may advantageously be chosen from ethanol, isopropanol, benzyl alcohol, glycerol, 1,2-propanediol (propylene glycol) and mixtures thereof.

The composition according to the invention may also comprise at least one or more standard cosmetic ingredients notably chosen from thickeners, gelling agents, which are both different from the above polysaccharides; sunscreens; anti-dandruff agents; antioxidants; chelating agents; reducing agents; oxidation bases, couplers, oxidizing agents, direct dyes; hair-straightening agents; nacreous agents and opacifiers; micas, nacres, glitter flakes; plasticizers or coalescers; pigments; fillers; fragrances; basifying or acidifying agents; silanes. A person skilled in the art will take care to select the ingredients included in the composition, and also the amounts thereof, so that they do not harm the properties of the compositions of the present invention.

The composition according to the invention can in addition advantageously comprise one or more silicone polymers derived from an amino acid, notably of following formula (A1) or (A2):

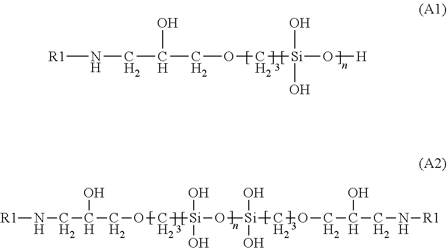

in which:
n is an integer of between 1 and 100, preferably between 1 and 50, more preferentially between 1 and 20, better still between 1 and 10, even better still between 1 and 5, even better still between 1 and 3,
R₁ is a polypeptide chain derived from an amino acid such as cysteine.

Preferably, the silicone polymer(s) derived from an amino acid correspond to formula (A1) in which R1 is a hydrolysed plant protein residue.

Mention may notably be made of the compounds having as INCI name: Hydrolyzed Wheat Protein PG-Propyl Silanetriol, Hydrolyzed Vegetable Protein PG-Propyl Silanetriol or Cystine Bis-PG-Propyl Silanetriol, which are sold under the names Crodasone® W, Crodasone® W PF, Keravis® PE or Crodasone® Cystine by Croda.

Preferably, the silicone polymer(s) derived from an amino acid can be present in the composition according to the invention at a content which can range from 0.1% to 2% by weight, notably from 0.2% to 1% by weight, relative to the total weight of the composition.

Better still, the silicone polymer(s) derived from an amino acid of formula (A1) in which R1 is a hydrolysed plant protein residue can be present in the composition according to the invention at a content which can range from 0.1% to 2% by weight, notably from 0.2% to 1% by weight, relative to the total weight of the composition.

According to a preferred embodiment of the invention, the cosmetic composition, preferably hair composition, may comprise:
one or more compounds of amino acid type corresponding to formula (I) as defined above, in which p=2 and R represents a hydrogen atom or a saturated, linear or branched, (C₁-C₄)alkyl group, optionally interrupted with a —S— heteroatom and/or optionally substituted with one or two groups chosen from hydroxyl, amino or —NH—C(NH)—NH₂; better still, R represents a hydrogen atom;
preferably present in a total content of at least 0.8% by weight, relative to the total weight of the composition, notably from 0.8% to 10% by weight, notably from 0.9% to 8% by weight, better still from 0.95% to 7% by weight, or even from 1% to 6% by weight, relative to the total weight of the composition;
one or more hydroxylated polycarboxylic acids, comprising from 4 to 6 carbon atoms, from 1 to 3 OH groups and 3 COOH groups, and/or salts thereof, preferably present in a total content of at least 0.5% by weight, relative to the total weight of the composition, better still from 0.5% to 10% by weight, notably from 1% to 8% by weight, better still from 1.2% to 6% by weight, even better still from 1.5% to 5% by weight, relative to the total weight of the composition;
one or more polysaccharides, notably chosen, alone or as a mixture, from cationic celluloses; cationic galactomannan gums, notably cationic guar gums; nonionic guar gums optionally modified with (poly)hydroxy(C₁-C₆)alkyl, notably hydroxypropyl, groups; and/or nonionic celluloses, which may or may not be substituted, such as nonionic cellulose ethers such as (C₁-C₄)alkylcelluloses and (poly)hydroxy(C₁-C₄)alkylcelluloses; the composition comprising one or more galactomannans; preferably in a total amount ranging from 0.01% to 10% by weight, better still from 0.05% to 5% by weight, even better still from 0.1% to 2% by weight, relative to the total weight of the composition;
optionally one or more nonionic surfactants; preferably present in a total content ranging from 0.05% to 10% by weight, preferably from 0.1% to 5% by weight, preferentially from 0.2% to 3% by weight, relative to the total weight of the composition according to the invention;
optionally one or more cationic surfactants; preferably present in a total amount ranging from 0.1% to 15% by weight, better still from 0.2% to 10% by weight, preferentially from 0.5% to 8% by weight, even better still from 1% to 6% by weight, relative to the total weight of the composition.

The cosmetic composition according to the invention notably finds a particularly advantageous application in the haircare field, notably for cleansing and/or conditioning the hair. The haircare compositions are preferably lotions, masks or conditioners, but may also be in the form of a shampoo, notably a conditioner, which may be rinsed out.

The cosmetic composition may or may not be rinsed out after being applied to the keratin materials, notably the hair. Rinsing may thus optionally be performed, for example with water after an optional leave-on time. Preferably, it is rinsed out after an optional leave-on time.

The subject of the invention is also a cosmetic treatment process, notably a hair treatment process, in particular for washing and/or conditioning keratin fibres, notably the hair, comprising the application to said fibres of a cosmetic composition according to the invention, preferably followed by rinsing after a possible leave-on time.

It is preferably a hair treatment process, in particular for washing and/or conditioning hair which has been sensitized, weakened and/or damaged, or else hair laden with metal ions, notably copper and/or calcium.

Said process may also comprise at least two successive steps of applying a composition as defined above to the hair, in particular to hair which has been sensitized, weakened and/or damaged; this is then referred to as a multi-application process.

A subject of the invention is also the use of the cosmetic composition according to the invention for strengthening hair which has notably been sensitized, weakened and/or damaged; and/or for limiting the loss of sheen of hair which has notably been sensitized, weakened and/or damaged.

A subject of the invention is also a process for the cosmetic treatment of the hair, notably hair which has been sensitized, weakened and/or damaged, comprising:
a step (i) of washing the hair, comprising the application of a washing cosmetic composition A comprising one or more anionic and/or amphoteric surfactants, followed by
a step (ii) of application to the hair of a cosmetic composition B as defined according to the present invention, followed by
a step (iii) of conditioning the hair, comprising the application of a cosmetic care composition C comprising one or more conditioning agents chosen from cationic surfactants, cationic polymers, silicones, fatty substances and mixtures thereof.

Step (i) of washing the hair may or may not be followed by a leave-on time, for example of 1 to 15 minutes, notably 2 to 5 minutes.

This step may or may not be followed by a rinsing step, for example with water, before step (ii) is performed.

Preferably, said step (i) is followed by a rinsing step, after a possible leave-on time of 1-15 minutes. The process according to the invention thus preferably comprises an intermediate rinsing step between steps (i) and (ii).

Step (ii) of application to the hair of a cosmetic composition according to the invention may or may not be followed by a leave-on time, for example of 1 to 15 minutes, notably 2 to 5 minutes.

This step may or may not be followed by a rinsing step, for example with water, before step (iii) is performed.

Preferably, said step (ii) is not followed by a rinsing step before step (iii) is performed.

Step (iii) of conditioning the hair may or may not be followed by a leave-on time, for example of 1 to 15 minutes, notably 2 to 5 minutes.

This step may or may not be followed by a rinsing step, for example with water. Preferably, said step (iii) is followed by a rinsing step, after a possible leave-on time of 1-15 minutes; and optionally by a drying step.

Steps (i), (ii) and (iii) are performed successively, or sequentially, which means that step (i) comes before step (ii), which itself comes before step (iii); intermediate steps, for example leave-on and/or rinsing and/or drying steps, may be present between each of these steps.

Preferably, steps (i) and (ii) are spaced apart by a maximum of one hour, better still 1 to 50 minutes, or even 5 to 45 minutes.

Preferably, steps (ii) and (iii) are spaced apart by a maximum of one hour, better still 1 to 50 minutes, or even 5 to 45 minutes.

A subject of the invention is also a process for the cosmetic treatment of the hair, notably hair which has been sensitized, weakened and/or damaged, comprising at least two successive steps of applying a cosmetic composition as defined according to the invention.

In other words, the composition is applied to the hair several times successively, that is to say that the steps of applying the composition according to the invention are performed one after the other with or without an intermediate rinsing step. Preferentially, the process according to the invention involves a step of rinsing the hair at least between two successive steps of applying the composition.

The leave-on time of the composition according to the invention on the hair can vary between several seconds (10 seconds for example) and several minutes (5 to 60 minutes for example), preferably between 1 and 30 minutes, better still between 2 and 15 minutes.

The application to the hair may be performed for example by means of a comb, a fine brush, with the aid of a coarse brush or with the fingers.

Between each successive step of applying the composition, the hair may be rinsed and/or may be dried.

The rinsing may be performed with water or a shampoo, after a possible leave-on time. The hair can then be squeezed dry, optionally washed using a standard shampoo and then dried.

The rinsing step may also be followed by a drying step, for example at room temperature (25° C.) or at a temperature greater than 40° C. Preferably, the hair is dried, in addition to a supply of heat, with a flow of air. During drying, a mechanical action may be exerted on the locks, such as combing, brushing, or running the fingers through. The drying step of the process of the invention may be performed with a hood, a hairdryer or a straightening iron. When the drying step is performed with a hood or a hairdryer, the drying temperature is between 40° C. and 110° C., preferably between 50° C. and 90° C. When the drying step is performed with a straightening iron, the drying temperature is between 110° C. and 220° C., preferably between 130° C. and 200° C.

The hair treatment process according to the invention may comprise more than two successive steps of applying the composition as defined above to the hair, for example at least 3 successive steps, or even at least 4 successive steps. These successive applications may also be performed over several weeks, or even several months.

The time between each application step may advantageously be between 1 day and several days, for example 1 day to 7 days, which corresponds to a weekly application of the composition, or between 2 and 4 days, which corresponds to a twice-weekly application of the composition.

The examples that follow serve to illustrate the invention without, however, being limiting in nature. In the examples that follow, unless otherwise indicated, all the amounts are indicated as mass percentages of active material (g % AM) relative to the total weight of the composition.

### EXAMPLE 1

The following compositions according to the invention were prepared from the ingredients shown in the tables below (g % AM):

TABLE 1

| Composition | A | B |
|---|---|---|
| Citric acid | 3.0 | 1.6 |
| Glycine | 5 | 3 |
| Sodium citrate | | 1.4 |
| Behentrimonium chloride | | 3.5 |
| Cetrimonium chloride | 1 | |
| Hydroxypropyl guar | 0.2 | 0.2 |
| Hydroxypropyl guar hydroxypropyltrimonium | 0.2 | |
| chloride | | |
| Polyquaternium-10 | | 0.3 |
| Quaternium-80 | 0.4 | |
| Hydroxypropyltrimonium hydrolysed wheat | 1 | |
| protein | | |
| Cetearyl alcohol | | 7 |
| Stearyl alcohol | | 1.8 |
| Cetyl esters | | 1.5 |
| Isopropyl alcohol | | 0.8 |
| Glycerol | 15 | 5 |
| PEG-40 hydrogenated castor oil | 0.8 | |
| Polysorbate 20 | 0.8 | 0.25 |
| Polysorbate 80 | 0.8 | |
| Propylene glycol | 5 | |
| Preserving agents | qs | qs |
| Water | qs 100% | qs 100% |

Composition A is in the form of a lotion and may advantageously be used, without rinsing, before shampooing or else after shampooing and before using a mask or a hair conditioner without being rinsed.

This composition may be used for disentangling of the hair, with the provision of care, and strength (a little less suppleness than conventional care treatments, body, and a mass effect). The composition also makes it possible to strengthen the hair.

Composition B is in the form of a cream which may advantageously be used in rinse-out mode after or before shampooing. This composition may be used for disentangling the hair, with the provision of care and strength (slightly less suppleness than conventional care treatments, body, and a mass effect). The composition may also be used for strengthening the hair.

### EXAMPLE 2

Composition C according to the invention and comparative composition C′ below were prepared from the ingredients shown in the tables below (g % AM):

TABLE 2

| Composition | C | C′ |
|---|---|---|
| Citric acid | 2 | — |
| Glycine | 1 | — |
| Behentrimonium Chloride | 3 | 3 |
| Hydroxypropyl guar | 0.15 | 0.15 |
| Aminopropyl dimethicone | 0.4 | 0.4 |
| Bis(C13-15 alkoxy) PG-amodimethicone | 0.3 | 0.3 |
| PEG-150/decyl alcohol/SMDI copolymer | 0.15 | 0.15 |
| Butyrospermum parkii (shea) butter | 0.5 | 0.5 |
| Cetearyl alcohol | 7 | 7 |
| Cetyl esters | 1.5 | 1.5 |
| Isododecane | 0.4 | 0.4 |
| Isopropyl alcohol | 0.7 | 0.7 |
| Glycerol | 1 | 1 |
| Polyquaternium-37 | 0.1 | 0.1 |
| Propylene glycol | 0.2 | 0.2 |
| Steareth-100 | 0.25 | 0.25 |
| Preserving agents | qs | qs |
| Water | qs 100% | qs 100% |

Composition C is in the form of a cream and may advantageously be used in rinse-out mode after or before shampooing. This composition may be used for disentangling the hair, with the provision of care and strength (provision of body and a mass effect). The composition also has hair strengthening properties.

The strengthening is measured using the DSC technique.

### (i) Preparation of the Locks

The measurements are performed on locks that have previously been bleached manually and then treated five times according to the following protocol: the lock is washed with a neutral shampoo, then rinsed, 2 g of the composition to be tested are applied to each 5.7 g lock of hair, the treatment is left on for 5 minutes, then rinsed again.

### (ii) Measurement Method

The differential scanning calorimetry (DSC) technique is known to those skilled in the art as a method for quantifying the strengthening of proteins in the cortex of keratin fibres (Kinetics of the changes imparted to the main structural components of human hair by thermal treatment, https://doi.org/l0.1016/j.tca.2018.01.014 & F.-J. Wortmann and H. Deutz, J. Appl. Polym Sci., 48, 137 (1993). The principle of the test is to measure the protein denaturing temperature. It is widely acknowledged that the higher the protein denaturing temperature, the better the integrity of the proteins of the cortex, which reflects the reduction in fibre breakage.

The denaturing temperature is directly linked to the bonding density of the keratin proteins present in the cortex. Thus, the lower the denaturing temperature, the lower the bonding density between the proteins: the disulfide bridges break and the cortex is damaged. A difference of 2° C. is acknowledged by those skilled in the art as a significant modification.

The machine used for taking the measurements is a TA Instruments DSC Q20 reference instrument. This machine measures the energy flow during heating of the sample. The temperature of maximum energy flow represents the denaturing temperature.

(iii) Results

The results of the denaturing temperature (Td) measurements for each of the locks treated according to the protocol described previously are summarized in the table below and correspond to the mean of three measurements taken per lock.

TABLE 3

| | Td (° C.) | SD |
|---|---|---|
| Natural lock (before bleaching treatment) | 153.31 | 1.36 |
| Untreated bleached lock | 138.73 | 0.75 |
| Bleached lock treated 5 times with composition C | 155.80 | 0.17 |
| Bleached lock treated 5 times with composition C′ | 142.12 | 0.41 |

*SD = standard deviation

These results show that the use of the composition according to the invention increases the bonding density of the keratin proteins presents in the cortex of the treated hair, thus enabling repair of the damaged hair.

Moreover, the denaturing temperature for the locks treated according to the present invention is better than that measured for natural and undamaged hair, thus showing that the hair has been repaired.

### EXAMPLE 3

The following compositions (A1 according to the invention and A2 comparative) were prepared from the ingredients shown in the table below (g % AM):

TABLE 4

| | A1 | A2 |
|---|---|---|
| | invention | comparative |
| GLYCINE | 3 | — |
| TAURINE | — | 3 |
| CITRIC ACID | 4 | 4 |
| PEG-150/DECYL ALCOHOL/SMDI | 0.6 | 0.6 |
| COPOLYMER | | |
| AMODIMETHICONE | 1.1 | 1.1 |
| BEHENTRIMONIUM CHLORIDE | 3 | 3 |
| HYDROXYPROPYL GUAR | 0.15 | 0.15 |
| CETEARYL ALCOHOL | 7 | 7 |
| CETYL ESTERS | 1.5 | 1.5 |
| ISOPROPYL ALCOHOL | 0.7 | 0.7 |
| GLYCERIN | 1 | 1 |
| POLYQUATERNIUM-37 | 0.08 | 0.08 |
| PROPYLENE GLYCOL | 0.2 | 0.2 |
| STEARETH-100 | 0.25 | 0.25 |
| Preservatives | Qs | Qs |
| Water | Qsp 100% | Qsp 100% |

Compositions A1 or A2 are applied to hair locks (moderately sensitized hair, SA20 type) weighing 5.4 g, previously washed with a classic shampoo (DOP), at the rate of 2.5 g of composition for 5.4 g of lock; the locks are kneaded 5 times. The locks are rinsed with water (21 passages between the fingers), wrung out (5 passages between the fingers), dried with a hair dryer then disentangled in order to be evaluated.

The performance in terms of smoothness was evaluated on dry hair, blind, after detangling, by 5 experts, on a scale ranging from 0 (very bad) to 5 (very good), in steps of 0.5.

The evaluation is tactile: the expert grasps the lock between the thumb and the index finger and slides his fingers along the lock from the upper part to the tips; it assesses whether the hair has rough edges and if it catches the fingers.

The results are the following:

TABLE 5

| | A1 | A2 |
|---|---|---|
| | (invention) | (comparative) |
| Expert 1 | 3 | 2.5 |
| Expert 2 | 3 | 2 |
| Expert 3 | 2.5 | 2 |
| Expert 4 | 3 | 2 |
| Expert 5 | 2.5 | 2 |
| Average | 2.8 | 2.1 |
| Standard deviation | 0.3 | 0.2 |

For all the experts, composition A1 was judged to be superior to composition A2 on the smoothness criterion.

The average value of the scores obtained shows an improvement in the smoothness to the touch criterion when composition A1 (according to the invention) is applied, compared to composition A2 (comparative). These results are significant regarding the standard deviations.

1-17. (canceled)

18. A composition, comprising:
(i) one or more amino acid compounds chosen from compounds of formula (I), salts thereof, or mixtures thereof: 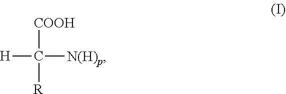
wherein p is an integer equal to 1 or 2,
wherein when p=1, R forms, with the nitrogen atom, a saturated heterocycle comprising from 5 to 8 ring members, optionally substituted with one or more groups chosen from hydroxyl or (C₁-C₄)alkyl, and
wherein when p=2, R represents a hydrogen atom or a saturated linear or branched (C₁-C₁₂)alkyl group, optionally interrupted with one or more heteroatoms or groups chosen from —S—, —NH—, or —C(NH)—, and/or optionally substituted with one or more groups chosen from hydroxyl (—OH), amino (—NH₂—), —SH, —COOH, —CONH₂ or —NH—C(NH)—NH₂;
wherein the total amount of (i) amino acid compounds chosen from compounds of formula (I), salts thereof, or mixtures thereof is at least 0.8% by weight, relative to the total weight of the composition;
(ii) one or more hydroxylated polycarboxylic acids, comprising from 2 to 8 carbon atoms and at least 3 COOH groups, salts thereof, or mixtures thereof; and
(iii) one or more polysaccharides chosen from galactomannans, derivatives thereof, or mixtures thereof.

19. The composition of claim 18, wherein p=2, and R represents a hydrogen atom or a saturated, linear or branched, (C₁-C₁₂)alkyl, optionally interrupted with one or more heteroatoms or groups chosen from —S—, —NH— or —C(NH)—, or optionally substituted with one or more groups chosen from hydroxyl (—OH), amino (—NH₂), —SH, —COOH, —CONH₂ or —NH—C(NH)—NH₂.

20. The composition of claim 18, wherein the (i) one or more amino acid compounds is chosen from glycine, proline, methionine, serine, arginine, lysine, salts thereof, or mixtures of two or more thereof.

21. The composition of claim 18, wherein the (i) one or more amino acid compounds is chosen from alkali metal salts, alkaline-earth metal salts, zinc salts, or mixtures of two or more thereof.

22. The composition of claim 18, wherein the total amount of (i) amino acid compound(s) chosen from compounds of formula (I), salts thereof, or mixtures thereof ranges from 0.8% to 10% by weight, relative to the total weight of the composition.

23. The composition of claim 18, wherein the (ii) one or more hydroxylated polycarboxylic acids is chosen from citric acid, salts thereof, or mixtures there.

24. The composition of claim 18, wherein the (ii) one or more hydroxylated polycarboxylic acids is sodium citrate.

25. The composition of claim 18, wherein the total amount of (ii) hydroxylated polycarboxylic acid(s) is at least 0.5% by weight, relative to the total weight of the composition.

26. The composition of claim 18, wherein the (iii) one or more galactomannans is chosen from nonionic galactomannans, cationic galactomannans, or mixtures thereof.

27. The composition of claim 18, wherein the (iii) one or more galactomannans is chosen from cationic guar gums, nonionic guar gums optionally modified with a (poly)hydroxy(C₁-C₆)alkyl groups, or mixtures of two or more thereof.

28. The composition of claim 18, wherein the (iii) one or more galactomannans is chosen from nonionic guar gums optionally modified with C₁-C₆ (poly)hydroxylalkyl groups or cationic polysaccharides.

29. The composition of claim 18, wherein the total amount of (iii) one or more polysaccharides, derivatives thereof, or mixtures thereof ranges from 0.01% to 10% by weight, relative to the total weight of the composition.

30. The composition of claim 18, wherein the (iii) one or more polysaccharides is chosen from nonionic surfactants, wherein the total amount of nonionic surfactants ranges from 0.05% to 10% by weight, relative to the total weight of the composition.

31. The composition of claim 18, further comprising (iv) one or more cationic surfactants, wherein the total amount of (iv) cationic surfactant(s) ranges from 0.1% to 15% by weight, relative to the total weight of the composition.

32. The composition of claim 18, further comprising (v) one or more non-silicone fatty substances, wherein the total amount of (v) non-silicone fatty substance(s) ranges from 0.1% to 20% by weight, relative to the total weight of the composition.

33. The composition of claim 18, further comprising (vi) water, wherein the total amount of (vi) water ranges from 55% to 90% by weight, relative to the total weight of the composition.

34. The composition of claim 18, wherein the composition is a composition for strengthening and/or for limiting the loss of sheen of hair which has been sensitized, weakened and/or damaged.

35. A method for treating keratin fibers, comprising:
(a) applying to the keratin fibers a composition comprising:
(i) one or more amino acid compounds chosen from compounds of formula (I), salts thereof, or mixtures thereof: 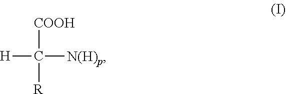
wherein p is an integer equal to 1 or 2,
wherein when p=1, R forms, with the nitrogen atom, a saturated heterocycle comprising from 5 to 8 ring members, optionally substituted with one or more groups chosen from hydroxyl or (C₁-C₄)alkyl, and
wherein when p=2, R represents a hydrogen atom or a saturated linear or branched (C₁-C₁₂)alkyl group, optionally interrupted with one or more heteroatoms or groups chosen from —S—, —NH—, or —C(NH)—, and/or optionally substituted with one or more groups chosen from hydroxyl (—OH), amino (—NH₂—), —SH, —COOH, —CONH₂ or —NH—C(NH)—NH₂;
wherein the total amount of (i) amino acid compounds chosen from compounds of formula (I), salts thereof, or mixtures thereof is at least 0.8% by weight, relative to the total weight of the composition;
(ii) one or more hydroxylated polycarboxylic acids, comprising from 2 to 8 carbon atoms and at least 3 COOH groups, salts thereof, or mixtures thereof; and
(iii) one or more polysaccharides chosen from galactomannans, derivatives thereof, or mixtures thereof; and
(b) rinsing the keratin fibers after a leave-on time.

36. A method for treating hair, comprising:
(a) washing the hair by applying to the hair a washing cosmetic composition A comprising one or more anionic and/or amphoteric surfactants;
(b) applying to the hair a cosmetic composition B comprising:
(i) one or more amino acid compounds chosen from compounds of formula (I), salts thereof, or mixtures thereof: 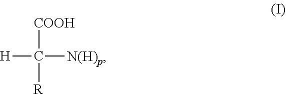
wherein p is an integer equal to 1 or 2,
wherein when p=1, R forms, with the nitrogen atom, a saturated heterocycle comprising from 5 to 8 ring members, optionally substituted with one or more groups chosen from hydroxyl or (C₁-C₄)alkyl, and
wherein when p=2, R represents a hydrogen atom or a saturated linear or branched (C₁-C₁₂)alkyl group, optionally interrupted with one or more heteroatoms or groups chosen from —S—, —NH—, or —C(NH)—, and/or optionally substituted with one or more groups chosen from hydroxyl (—OH), amino (—NH₂—), —SH, —COOH, —CONH₂ or —NH—C(NH)—NH₂;
wherein the total amount of (i) amino acid compounds chosen from compounds of formula (I), salts thereof, or mixtures thereof is at least 0.8% by weight, relative to the total weight of the composition;
(ii) one or more hydroxylated polycarboxylic acids, comprising from 2 to 8 carbon atoms and at least 3 COOH groups, salts thereof, or mixtures thereof; and
(iii) one or more polysaccharides chosen from galactomannans, derivatives thereof, or mixtures thereof; and
(c) conditioning the hair by applying to the hair a cosmetic care composition C comprising one or more conditioning agents chosen from cationic surfactants, cationic polymers, silicones, fatty substances, or mixtures of two or more thereof.

37. The method of claim 36, wherein cosmetic composition B is a composition for strengthening and/or for limiting the loss of sheen of hair which has been sensitized, weakened and/or damaged.
